(19) United States
(12) Patent Application Publication
(10) Pub. No.: US 2015/0079136 A1
(43) Pub. Date: Mar. 19, 2015

(54) CONTROLLED RELEASE PHARMACEUTICAL FORMULATIONS OF DIRECT THROMBIN INHIBITORS

(71) Applicant: RUBICON RESEARCH PRIVATE LIMITED, Mumbai (IN)

(72) Inventors: Pratibha S. Pilgaonkar, Mumbai (IN); Maharukh T. Rustomjee, Mumbai (IN); Anilkumar S. Gandhi, Mumbai (IN)

(21) Appl. No.: 14/391,030

(22) PCT Filed: Apr. 10, 2013
(86) PCT No.: PCT/IN2013/000236

§ 371 Date: Oct. 7, 2014

(30) Foreign Application Priority Data
Apr. 10, 2012 (IN) 1161/MUM/2012

(51) Int. Cl. A61K 9/20; A61K 47/38; A61K 47/02; A61K 47/12; A61K 47/34; A61K 47/26; A61K 31/4439; A61K 47/32

(52) U.S. Cl. CPC A61K 9/2086; A61K 31/4439; A61K 47/38; A61K 47/32; A61K 47/12; A61K 47/34; A61K 47/26; A61K 47/02
USPC 424/400; 514/338

(57) ABSTRACT

The present invention relates to controlled release pharmaceutical formulations of direct thrombin inhibitors and processes for preparing such compositions. Particularly the present invention relates to oral controlled release pharmaceutical compositions comprising dabigatran etexilate or pharmaceutically acceptable salts thereof.

### FIELD OF THE INVENTION

The present invention relates to controlled release pharmaceutical formulations of direct thrombin inhibitors and processes for preparing such compositions. Particularly the present invention relates to oral controlled release pharmaceutical compositions comprising dabigatran etexilate or pharmaceutically acceptable salts thereof.

### BACKGROUND OF THE INVENTION

Atrial fibrillation is the most common cardiac arrhythmia which is characterized by abnormal heart rhythm. It is considered to be a common cause of irregular heart beat and can cause stroke and other systemic embolic events, eventually leading to death. It has been seen that the incidence of atrial fibrillation increases with age and nearly 6% of individuals over the age of 65 are affected while the prevalence is about 8% in individuals over the age of 80. The lack of organized cardiac contractions in atrial fibrillation generally results in some stagnant blood in the left atrium or left atrial appendage. This lack of movement of blood leads to thrombus formation or blood clotting. Patients with atrial fibrillation are therefore at greater risk of developing clots which increases the risk of stroke and other systemic embolic events. Since the consequence of stroke or systemic embolism is devastating, a primary aim of therapy for atrial fibrillation is to reduce the risk of arterial thrombus formation and thromboembolism. Anticoagulants such as warfarin are mainly used in case of atrial fibrillation along with other medications such as beta blockers and calcium channel blockers or some noninvasive rhythm control methods. Though anticoagulation therapy with warfarin has been shown to significantly reduce the incidence of stroke or systemic embolism, its use is found to be cumbersome due to multiple diet and drug interactions, chances of hemorrhage which are difficult to manage, requirement of frequent laboratory monitoring etc. Use of a newer safe and effective anticoagulant is therefore necessary.

Direct thrombin inhibitors, is another class of anticoagulants that act by directly inhibiting the enzyme thrombin and are expected to replace heparin (and derivatives) and warfarin in various clinical scenarios. Thrombin, a serine protease protein formed by proteolytic cleavage of prothrombin, converts soluble fibrinogen into insoluble strands of fibrin and further catalyzes many other coagulation-related reactions. Direct thrombin inhibitors inhibit thrombin including fibrin-bound thrombin, thereby delimiting thrombus growth, provide predictable anticoagulant responses because they are not bound to plasma proteins and have no drug-drug interactions. Depending on their interaction with the thrombin molecule, there are bivalent as well as univalent types of direct thrombin inhibitors, with some being in clinical use, while others undergoing clinical development.

Dabigatran is a potent, reversible, univalent direct thrombin inhibitor. It reduces the risk of stroke and systemic embolism in patients with non-valvular atrial fibrillation. It is also useful in primary prevention of venous thromboembolic events in adult patients who have undergone elective total hip replacement surgery or total knee replacement surgery. Dabigatran inhibits free thrombin, fibrin-bound thrombin and thrombin-induced platelet aggregation. Dabigatran was first disclosed in WO98/37075, which claimed compounds with a thrombin-inhibiting effect and the effect of prolonging the thrombin time, under the name 1-methyl-2-[N-[4-(N-n-hexyloxycarbonylamidino) phenyl]aminomethyl]benzimidazol-5-ylcarboxylic acid-N-(2-pyridyl)-N-(2 ethoxycarbonylethyl)amides.

Dabigatran is currently available as dabigatran etexilate mesylate (DEM) in Europe and United States under the brand name Pradaxa® from Boehringer Ingelheim as immediate release oral capsules containing pellets of 75 mg, 110 mg and 150 mg and 75 mg and 150 mg strengths respectively to be administered twice daily. DEM is a salt form of the prodrug dabigatran etexilate which after oral administration is rapidly absorbed and converted to dabigatran by esterase-catalyzed hydrolysis in the liver. U.S Patent Application 200610183779A1 describes the marketed formulation of DEM in the form of pellets that comprise tartaric acid cores coated with active layer coating with a separating agent layer separating the acid core from the active substance containing layer.

DEM is a yellow-white to yellow non-hygroscopic powder that exists in two anhydrous polymorphic forms, Form I and II. The aqueous solubility of DEM is strongly pH dependent with rather high solubility in acidic media and very poor solubility in neutral and basic media while solubility in water is 1.8 mg/mL. Therefore, dabigatran etexilate is absorbed better in an acidic milieu in the gastrointestinal tract. However, at higher pH in intestine where solubility is low, absorption tends to be poor and erratic. DEM is BCS Class II drug, indicating poor aqueous solubility but good membrane permeability. DEM is stable in the solid state and not sensitive to light irradiation but it predominantly undergoes degradation by hydrolytic pathways in the presence of moisture. It is also acid sensitive. The elimination half life is 12-17 hours with single dosing and decreases to about 8 hours upon multiple dosing. Dabigatran etexilate is a substrate of the efflux transporter P-glycoprotein. After oral administration of dabigatran etexilate in healthy volunteers, Cmaxoccurs at 1 hour post-administration in the fasted state. Coadministration with a high-fat meal delays the time to Cmaxby approximately 2 hours but has no effect on the bioavailability of dabigatran.

The absolute bioavailability of dabigatran following oral administration of dabigatran etexilate is approximately about 3-7%. However, the oral bioavailability of dabigatran etexilate from the marketed formulation increases by 75% when the pellets are taken without the capsule shell as compared to the intact capsule formulation. Without being bound to any theory, the low bioavailability of DEM is primarily because the active agent is unavailable for absorption as the dosage form passes down the gastrointestinal tract, resulting in precipitation of the drug therein in the intestinal region where its solubility is low than in acidic milieu. Moreover, dabigatran etexilate also undergoes P-glycoprotein mediated efflux, which further limits systemic absorption of the active and its bioavailability.

Additionally, pharmaceutically active agents which exhibit low bioavailability unfortunately create a need for frequent dosing of a large amount of pharmaceuticals in order to provide and maintain therapeutic levels. However, the need for multiple dosings in a day, present patient compliance problems and also cause fluctuations in serum concentrations of the active agents and toxicity. Furthermore direct thrombin inhibitors such as dabigatran etexilate have low therapeutic index and therefore fluctuations in serum concentrations of these agents due to multiple dosings can reduce the safety and efficacy of these agents and increase side effects such as increased risk of bleeding.

Need thus exists for controlled release dosage form of direct thrombin inhibitors such dabigatran etexilate or pharmaceutically acceptable salts thereof that would enable better patient compliance and offer advantages over conventional immediate release formulations. Controlled release formulations would also lessen or prevent potentially undesirable effects by reducing peak blood levels (Cmax) and increase drug efficacy (Cmin) by maintaining desired therapeutic plasma concentrations for longer period. Need also exists to address the low bioavailability issue of direct thrombin inhibitors and develop dosage forms thereof with desired, high or improved and reproducible bioavailability. Particularly, need exists for controlled release formulations of direct thrombin inhibitors with improved solubility, absorption and bioavailability. Further, direct thrombin inhibitor such as dabigatran etexilate mesylate is sensitive to acid and moisture and therefore need also exists to develop controlled release formulations thereof that are stable over the shelf life.

Attempts have not been made by researchers to provide controlled release compositions of direct thrombin inhibitors. Attempts have also not been made by researchers to provide controlled release formulations of direct thrombin inhibitors with improved solubility, absorption and bioavailability.

The present inventors after rigorous experimentation provide controlled release formulations of direct thrombin inhibitors that not only release the active agent continuously in a predetermined manner and lessen the frequency of dosing but also reduce peak—trough fluctuations thereby maintaining desired therapeutic concentrations for longer duration of time and minimizing side effects otherwise associated immediate release tablets. The present inventors further provide controlled release formulations of direct thrombin inhibitors with improved solubility and bioavailability of the active agent.

Furthermore since direct thrombin inhibitor such as dabigatran etexilate mesylate is a substrate of efflux pump P-glycoproteins and has pH dependent solubility with high solubility in acidic media and very poor solubility in neutral and basic media, the present inventors provide controlled release formulations of direct thrombin inhibitor in the form of gastroretentive dosage form. Such a dosage form continuously delivers the active at a predetermined rate in the upper regions of the gastrointestinal tract in an acid milieu where solubility of dabigatran etexilate mesylate is better resulting in improved absorption of the active agent and improved bioavailability. Such a dosage form also minimizes exposure of the drug to efflux pump P-glycoproteins thereby further improving bioavailability and efficacy. Such a dosage form may comprise solubilized active agent to further improve the bioavailability of the active agents.

The present inventors thus provide controlled release formulations comprising at least one direct thrombin inhibitor such as dabigatran etexilate mesylate, at least one release controlling agent and at least one pharmaceutically acceptable excipient. The formulations of the present invention are stable, easy or convenient to prepare, and provide the desired in vitro release and bioavailability.

### SUMMARY OF THE INVENTION

The present invention relates to controlled release pharmaceutical formulations of direct thrombin inhibitors. Particularly the present invention relates to oral controlled release pharmaceutical compositions comprising dabigatran etexilate or pharmaceutically acceptable salts thereof.

### DETAILED DESCRIPTION OF THE INVENTION

The present invention provides oral controlled release pharmaceutical compositions comprising at least one direct thrombin inhibitor, at least one release controlling agent and at least one pharmaceutically acceptable excipient.

The term “composition” or “formulation” or “dosage form” has been employed interchangeably for the purpose of the present invention and mean that it is a pharmaceutical composition which is suitable for administration to a patient or subject. The subject can be an animal, preferably a mammal, more preferably a human. For the purpose of the present invention terms “controlled release” or “sustained release” or “extended release” or “prolonged release” have been used interchangeably and mean broadly that the direct thrombin inhibitor is released at a predetermined rate that is slower than the immediate release formulation.

The term “direct thrombin inhibitor's” as employed herein refers to any compound that acts by directly inhibiting the enzyme thrombin, both free and fibrin-bound thrombin as well as thrombin-induced platelet aggregation; including, but not limited to, dabigatran, argatroban, inogatran, melagatran, ximelagatran, hirudin, bivalirudin, lepirudin, desirudin and the like, in the form of free acid or free base or pharmaceutically acceptable prodrugs, pharmaceutically acceptable salts, pharmaceutically acceptable salts of prodrugs, active metabolites, polymorphs, solvates, hydrates, enantiomers, optical isomers, precursors, derivatives, analogs, amorphous form, diastereomers, diastereomeric mixtures, tautomers or racemic mixtures thereof. In one embodiment, the direct thrombin inhibitors employed in the compositions of the present invention include, but are not limited to, univalent inhibitors such as, but not limited to, dabigatran, argatroban, melagatran, ximelagatran, and the like; or bivalent inhibitors such as, but not limited to hirudin, bivalirudin, lepirudin, desirudin and the like; and various combinations thereof in the form of free acid or free base or pharmaceutically acceptable prodrugs, pharmaceutically acceptable salts, pharmaceutically acceptable salts of prodrugs, active metabolites, polymorphs, solvates, hydrates, enantiomers, optical isomers, tautomers or racemic mixtures thereof.

Pharmaceutically effective amount of direct thrombin inhibitor is employed in the composition of the present invention. The term “effective amount” refers to an amount effective to achieve desired preventive, therapeutic and/or beneficial effect, such as but not limited to reducing the risk of stroke and systemic embolism in patients with non-valvular atrial fibrillation or preventing venous thromboembolic events in adult patients who have undergone elective total hip replacement surgery or total knee replacement surgery, and the like. In one embodiment the amount of direct thrombin inhibitor in the composition can vary from about 0.01 weight % to about 90 weight %, based on the total weight of the composition. In another embodiment the amount of direct thrombin inhibitor in the composition can vary from about 0.02 weight % to about 85 weight %, based on the total weight of the composition. In still another embodiment, the amount of direct thrombin inhibitor in the composition can vary from about 0.05 weight % to about 80 weight %, based on the total weight of the composition. In one embodiment the compositions of the present invention may be administered at a dose of about 0.01 mg to about 400 mg of direct thrombin reductase inhibitor. In another embodiment the compositions of the present invention may be administered at a dose of about 0.1 mg to about 350 mg of direct thrombin inhibitor. In still another embodiment the compositions of the present invention may be administered at a dose of about 0.2 mg to about 300 mg of direct thrombin inhibitor. In one embodiment, the direct thrombin inhibitor employed for the present invention is dabigatran in the form of free acid or free base or pharmaceutically acceptable prodrugs, pharmaceutically acceptable salts, pharmaceutically acceptable salts of prodrugs, active metabolites, polymorphs, solvates, hydrates, enantiomers, optical isomers, tautomers or racemic mixtures thereof. In a further embodiment, the direct thrombin inhibitor employed in the present invention is dabigatran etexilate mesylate.

The controlled release compositions of the present invention comprise along with at least one direct thrombin inhibitor, at least one release controlling agent. The term “release controlling agent” as used herein means any excipient that can retard the release of active agent and includes, but is not limited to, polymeric release controlling agent, non-polymeric release controlling agent or combinations thereof.

Suitable polymeric release controlling agent may be employed in the compositions of the present invention. In one embodiment, the polymeric release controlling agent that may be employed in the compositions of the present invention may be pH independent or pH dependent or any combination thereof. In another embodiment, the polymeric release controlling agent employed in the compositions of the present invention may be swelling or non-swelling. In a further embodiment, polymeric release controlling agents that may be employed in the compositions of the present invention include, but are not limited to, cellulose derivatives, saccharides or polysaccharides, poly(oxyethylene)-poly(oxypropylene) block copolymers (poloxamers), vinyl derivatives or polymers or copolymers thereof, polyalkylene oxides and derivatives thereof, maleic copolymers, acrylic acid derivatives or the like or any combinations thereof.

Cellulose derivatives include, but are not limited to, ethyl cellulose, methylcellulose, hydroxypropylmethylcellulose (HPMC), hydroxypropyl cellulose (HPC), hydroxyethyl cellulose, hydroxymethyl cellulose, hydroxypropyl ethylcellulose, carboxymethylethyl cellulose, carboxy ethylcellulose, carboxymethyl hydroxyethylcellulose, hydroxyethylmethyl carboxymethyl cellulose, hydroxyethyl methyl cellulose, carboxymethyl cellulose, methylhydroxyethyl cellulose, methylhydroxypropyl cellulose, carboxymethyl sulfoethyl cellulose, sodium carboxymethyl cellulose, cellulose acetate, cellulose acetate phthalate, cellulose acetate butyrate, hydroxypropylmethylcellulose acetate succinate, hydroxypropylmethylcellulose phthalate, hydroxymethyl ethylcellulose phthalate, cellulose acetate phthalate, cellulose acetate succinate, cellulose acetate maleate, cellulose acetate trimelliate, cellulose benzoate phthalate, cellulose propionate phthalate, methylcellulose phthalate, ethylhydroxy ethylcellulose phthalate, or combinations thereof.

Saccharides or polysaccharides include, but are not limited to, guar gum, xanthan gum, gum arabic, tragacanth or combinations thereof. Vinyl derivatives, polymers and copolymers thereof include, but are not limited to, polyvinylacetate aqueous dispersion (Kollicoat® SR 30D), copolymers of vinyl pyrrolidone, copolymers of polyvinyl alcohol, mixture of polyvinyl acetate and polyvinylpyrrolidone (e.g. Kollidon® SR), polyvinyl alcohol phthalate, polyvinylacetal phthalate, polyvinyl butylate phthalate, polyvinylacetoacetal phthalate, polyvinylpyrrolidone (PVP), or combinations thereof. Polyalkylene oxides and derivatives thereof include, but are not limited to, polyethylene oxide and the like or any combinations thereof.

Acrylic acid derivatives include, but are not limited to, methacrylic acids, polymethacrylic acids, polyacrylates, especially polymethacrylates like a) copolymer formed from monomers selected from methacrylic acid, methacrylic acid esters, acrylic acid and acrylic acid esters b) copolymer formed from monomers selected from butyl methacrylate, (2-dimethylaminoethyl)methacrylate and methyl methacrylate c) copolymer formed from monomers selected from ethyl acrylate, methyl methacrylate and trimethylammonioethyl methacrylate chloride or d) copolymers of acrylate and methacrylates with/without quarternary ammonium group in combination with sodium carboxymethylcellulose, e.g. those available from Röhm GmbH under the trademark Eudragit® like Eudragit EPO (dimethylaminoethyl methacrylate copolymer; basic butylated methacrylate copolymer), Eudragit RL and RS (trimethylammonioethyl methacrylate copolymer), Eudragit NE30D and Eudragit NE40D (ethylacrylate methymethacrylate copolymer), Eudragit® L 100 and Eudragit® S (methacrylic acid methyl methacrylate copolymer), Eudragit® L 100-55 (methacrylic acid ethyl acrylate copolymer), Eudragit RD 100 (ammoniomethacrylate copolymer with sodium carboxymethylcellulose); or the like or any combinations thereof. Maleic copolymer based polymeric release controlling agent includes, but is not limited to, vinylacetatemaleic acid anhydride copolymer, styrenemaleic acid anhydride copolymer, styrenemaleic acid monoester copolymer, vinylmethylether maleic acid anhydride copolymer, ethylene maleic acid anhydride copolymer, vinylbutylether maleic acid anhydride copolymer, acrylonitrilemethyl acrylatemaleic acid anhydride copolymer, butyl acrylatestyrenemaleic acid anhydride copolymer and the like, or combinations thereof. In one embodiment, polymers with low viscosity are employed in the compositions of the present invention as release controlling agent such as, but not limited to, Methocel K4M, and the like or combinations.

The term “non-polymeric release controlling agent” as used herein refers to any excipient that can retard the release of an active agent and that does not comprise of repeating units of monomers. Suitable non-polymeric release controlling agents employed in the present invention include, but are not limited to, fatty acids, long chain alcohols, fats and oils, waxes, phospholipids, eicosonoids, terpenes, steroids, resins and the like or combinations thereof. Non-polymeric release controlling agents employed may be pH dependent or pH independent in nature.

Fatty acids are carboxylic acids derived from or contained in an animal or vegetable fat or oil. Fatty acids are composed of a chain of alkyl groups containing from 4 to 22 carbon atoms and are characterized by a terminal carboxyl group. Fatty acids that may be employed in the present invention include, but are not limited to, hydrogenated palm kernel oil, hydrogenated peanut oil, hydrogenated palm oil, hydrogenated rapeseed oil, hydrogenated rice bran oil, hydrogenated soybean oil, hydrogenated sunflower oil, hydrogenated castor oil, hydrogenated cottonseed oil, and the like, and mixtures thereof. Other fatty acids include, but are not limited to, decenoic acid, docosanoic acid, stearic acid, palmitic acid, lauric acid, myristic acid, and the like, and mixtures thereof. In one embodiment the fatty acids employed include, but are not limited to, hydrogenated palm oil, hydrogenated castor oil, stearic acid, hydrogenated cottonseed oil, palmitic acid, and mixtures thereof. Suitable long chain monohydric alcohols include, but are not limited to, cetyl alcohol, stearyl alcohol or mixtures thereof.

Waxes are esters of fatty acids with long chain monohydric alcohols. Natural waxes are often mixtures of such esters, and may also contain hydrocarbons. Waxes are low-melting organic mixtures or compounds having a high molecular weight and are solid at room temperature. Waxes may be hydrocarbons or esters of fatty acids and alcohols. Waxes that may be employed in the present invention include, but are not limited to, natural waxes, such as animal waxes, vegetable waxes, and petroleum waxes (i.e., paraffin waxes, microcrystalline waxes, petrolatum waxes, mineral waxes), and synthetic waxes. Specific examples include, but are not limited to, spermaceti wax, carnauba wax, Japan wax, bayberry wax, flax wax, beeswax, Chinese wax, shellac wax, lanolin wax, sugarcane wax, candelilla wax, paraffin wax, microcrystalline wax, petrolatum wax, carbowax, and the like, or mixtures thereof. Mixtures of these waxes with the fatty acids may also be used. Waxes are also monoglyceryl esters, diglyceryl esters, or triglyceryl esters (glycerides) and derivatives thereof formed from a fatty acid having from about 10 to about 22 carbon atoms and glycerol, wherein one or more of the hydroxyl groups of glycerol is substituted by a fatty acid. Glycerides that may be employed in the present invention include, but are not limited to, glyceryl monostearate, glyceryl distearate, glyceryl tristearate, glyceryl dipalmitate, glyceryl tripalmitate, glyceryl monopalmitate, glyceryl dilaurate, glyceryl trilaurate, glyceryl monolaurate, glyceryl didocosanoate, glyceryl tridocosanoate, glyceryl monodocosanoate, glyceryl monocaproate, glyceryl dicaproate, glyceryl tricaproate, glyceryl monomyristate, glyceryl dimyristate, glyceryl trimyristate, glyceryl monodecenoate, glyceryl didecenoate, glyceryl tridecenoate, glyceryl behenate, polyglyceryl diisostearate, lauroyl macrogolglycerides, oleyl macrogolglycerides, stearoyl macrogolglycerides, and the like, or mixtures thereof. Resins employed in the compositions of the present invention include, but are not limited to, shellac and the like or any combinations thereof.

In one embodiment the non-polymeric release controlling agent employed includes, but is not limited to, Cutina® (Hydrogenated castor oil), Hydrobase® (Hydrogenated soybean oil), Castorwax® (Hydrogenated castor oil, Croduret® (Hydrogenated castor oil), Carbowax®, Compritol® (Glyceryl behenate), Sterotex® (Hydrogenated cottonseed oil), Lubritab® (Hydrogenated cottonseed oil), Apifil® (Wax yellow), Akofine® (Hydrogenated cottonseed oil), Softisan® (Hydrogenated palm oil), Hydrocote® (Hydrogenated soybean oil), Corona® (Lanolin), Gelucire® (Macrogolglycerides Lauriques), Precirol® (Glyceryl Palmitostearate), Emulcire™ (Cetyl alcohol), Plurol® diisostearique (Polyglyceryl Diisostearate), Geleol® (Glyceryl Stearate), and mixtures thereof.

The amount of release controlling agent used in the controlled release formulations of the present invention may vary depending upon the degree of controlled or sustained release desired. In an embodiment, release controlling agent is present in the composition in an amount from about 1% to about 95% by weight of the dosage form. In another embodiment, release controlling agent is present in the formulation in an amount from about 2% to about 90% by weight of the dosage form. In a further embodiment, release controlling agent is present in the formulation in an amount from about 5% to about 85% by weight of the dosage form.

In one embodiment, the direct thrombin inhibitor in the form of, but not limited to, powder, granules, pellets, beads, minitablets or the like is treated with at least one release controlling agent. In a further embodiment the active agent may be in micronized form. The active ingredient may be treated by any of the techniques known in the art such as, but not limited to, melt granulation, hot melt extrusion, fluid bed coating, wet granulation, spray drying, extrusion-spheronization, dry granulation or roll compaction. Lipids or waxes can also be employed in the form of an aqueous dispersion stabilized by surfactants and suitable stabilizers. In one embodiment, the direct thrombin inhibitor is incorporated in the controlled release formulations of the present invention in the solubilized form. In another embodiment, the direct thrombin inhibitor or solubilized direct thrombin inhibitor is blended or physically mixed with release controlling agent. In one embodiment, the direct thrombin inhibitor or solubilized direct thrombin inhibitor when coated with a release controlling agent, coating may be carried out in the range from about 1% to about 80% weight gain, preferably from about 2% to about 60% weight gain, more preferably from about 5 to about 50% weight gain. In a further embodiment treated direct thrombin inhibitor or solubilized direct thrombin inhibitor is incorporated in the dosage forms of the present invention.

Controlled release of direct thrombin inhibitor may be accomplished by any means known in the pharmaceutical art, such as, but not limited to, matrix controlled-release systems, coated controlled release systems, coated-matrix controlled release systems, osmotic controlled-release systems, multiparticulate controlled-release systems, non-gastroretentive controlled release systems and the like.

In one embodiment the controlled release formulation of the present invention is in the form of a gastroretentive dosage form. For the purpose of the present invention the term “gastroretentive” or “gastric retention” or “gastroretention” or “retained in upper gastrointestinal tract” when used with respect to the dosage form of the present invention, means that the dosage form or at least a portion thereof remains in the upper gastrointestinal tract including stomach, for about 30 minutes or more. In another embodiment, the gastroretentive dosage form of the present invention remains in the upper gastrointestinal tract including stomach, for about 30 minutes to about 12 hours. In another embodiment controlled release formulation of the present invention is in the form of a gastroretentive dosage form for improved bioavailability. In a further embodiment, gastroretentive dosage forms that are retained in the upper gastrointestinal tract for a prolonged period of time after oral administration and release the active ingredient continuously at a predetermined rate or in a sustained manner are employed for delivering direct thrombin inhibitors that exhibit low oral bioavailability. Design of such gastroretentive dosage forms is a challenge for a formulator because of the complexities of physiological effects that have implications on drug release and absorption in vivo. The controlled release gastroretentive dosage forms of the present invention release the active at a predetermined rate and provide improved bioavailability when compared to conventional immediate release dosage forms.

The controlled release formulations of the present invention in addition to at least one direct thrombin inhibitor and at least one release controlling agent as discussed above, comprise at least one swelling agent. The controlled release formulations of the present invention in the form of a gastroretentive dosage form comprise in addition to at least one direct thrombin inhibitor and at least one release controlling agent as discussed above, at least one swelling agent. The swelling agents employed herein swell voluminously in the presence of gastric contents to increase the size of dosage form such that it precludes its passage through the pyloric sphincter thereby retaining the compositions of the present invention in the upper gastrointestinal tract. The controlled release gastroretentive formulations of the present invention comprise at least one direct thrombin inhibitor, at least one release controlling agent, at least one swelling agent and at least one pharmaceutically acceptable excipient.

The swelling agent used in the present invention includes, but is not limited to, one or more swellable biocompatible hydrophilic polymers. In one embodiment, the swelling agents are employed in the dry state or in a form that has substantial capacity for water uptake. Hydrophilic polymers used as swelling agents that are useful in preparation of the dosage forms of the present invention are polymers that are nontoxic and swell in a dimensionally unrestricted manner upon imbibing gastric fluid.

Suitable swelling agents employed in the dosage forms of the present invention include, but are not limited to, polyalkylene oxides; cellulosic polymers such as, but not limited to, hydroxypropyl cellulose, hydroxypropyl methyl cellulose, hydroxyethyl cellulose, sodium carboxy methylcellulose, methyl cellulose; acrylic acid and methacrylic acid polymers, and esters thereof, polyethylene oxide, maleic anhydride polymers; polymaleic acid; poly(acrylamides); carbopol, poly(olefinic alcohol)s; poly(N-vinyl lactams); polyols; polyoxyethylated saccharides; polyoxazolines; polyvinylamines; polyvinylacetates; polyimines; starch and starch-based polymers; polyurethane hydrogels; chitosan; polysaccharide gums such as xanthan gum; alginates; zein; shellac-based polymers; polyacrylic acid, maltodextrin, pre-gelatinized starch and polyvinyl alcohol, or mixtures thereof. In one embodiment, swelling agents of different viscosity grades can be incorporated in the compositions of the present invention to achieve gastroretention. In another embodiment, swelling agents of high viscosity can be incorporated in the compositions of the present invention to achieve gastroretention such as, but not limited to, Methocel K100M, Polyox WSR303, and the like or combinations thereof. In one embodiment, the swelling agent employed may function as a release controlling agent. In another embodiment, the swelling agent employed may be a swelling release controlling agent.

The amount of swelling agent employed in the controlled release gastroretentive dosage forms of the present invention is from about 2% to about 98% by weight of the final dosage form. In one embodiment, the weight percent of the swelling agent in the final dosage form is about 5% to about 95%. In another embodiment, the weight percent of the swelling agent in the final dosage form is about 10% to about 90%. The amount and type of swelling agents employed in the gastroretentive dosage forms of the present invention ensures that there is sufficient swelling for retention of the dosage form. In one embodiment, the controlled release dosage form is a multilayered gastroretentive tablet with drug layer comprising at least one direct thrombin inhibitor and at least one release controlling agent; and at least one gastroretentive layer/s comprising at least one swelling agent wherein the swelling agents ensure that there is sufficient swelling for retention of the dosage form despite erosion of the drug layer. These swelling agents ensure that within 2 hours at least two dimensions of the dosage form namely length and width is more than 8 mm, preferably more than 10 mm.

In addition to the above discussed excipients, the controlled release compositions of the present invention comprise at least one pharmaceutically acceptable excipients, such as, but not limited to, solubility enhancing agents, p-glycoprotein inhibitors, swelling enhancers, permeation enhancers, pH modifiers, binders, lubricants, diluents, disintegrants, glidants, stabilizers, preservatives, colorants and the like or combinations thereof.

In one embodiment the increase in instantaneous solubility of direct thrombin inhibitor is achieved by using at least one solubility enhancing agent. In another embodiment, the controlled release formulations of the present invention comprise solubilized direct thrombin inhibitor which comprises at least one direct thrombin inhibitor, at least one solubility enhancing agent and optionally at least one pharmaceutically acceptable excipient, such as, but not limited to diluents and the like. In one embodiment, the controlled release formulation of the present invention comprises at least one direct thrombin inhibitor, at least one release controlling agent, at least one solubility enhancing agent, and at least one pharmaceutically acceptable excipient. In a further embodiment, the controlled release gastroretentive dosage form of the present invention comprises at least one direct thrombin inhibitor, at least one release controlling agent, at least one swelling agent, at least one solubility-enhancing agent, and at least one pharmaceutically acceptable excipient.

The solubility enhancing agent or solubilizer that may be employed in the compositions of the present invention may include one or more surfactant, complexing agent, hydrotropic agent, ion pairing agent and the like or combinations thereof. The solubility enhancing agent as employed in the present invention includes, but is not limited to, hydrophilic surfactants, lipophilic surfactants and the like or mixtures thereof. The surfactants employed in the present invention may also include, but are not limited to, ionic surfactants comprising cationic or anionic surfactants, zwitterionic or amphiphilic surfactants or nonionic surfactants or the like or any combinations thereof. The ionic surfactants include, but are not limited to, alkylammonium salts; fusidic acid salts; fatty acid derivatives of amino acids, oligopeptides, or polypeptides; glyceride derivatives of amino acids; lecithins or hydrogenated lecithins; lysolecithins or hydrogenated lysolecithins; phospholipids or derivatives thereof; lysophospholipids or derivatives thereof; carnitine fatty acid ester salts; salts of alkylsulfates; sodium lauryl sulphate, fatty acid salts; sodium docusate; acyl lactylates; mono- or di-acetylated tartaric acid esters of mono- or di-glycerides; succinylated mono- or di-glycerides; citric acid esters of mono- or di-glycerides; or mixtures thereof. The amphiphilic surfactants include, but are not limited to, d-α-tocopheryl polyethylene glycol 1000 succinate (Vitamin E TPGS) and d-α-tocopherol acid salts such as succinate, acetate, etc. The non-ionic surfactants include, but are not limited to, fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols or sterol derivatives; polyethylene glycol alkyl ethers; sugar esters; sugar ethers; lactic acid derivatives of mono- or di-glycerides; oil-soluble vitamins/vitamin derivatives; PEG fatty acid esters; polyglycerized fatty acid; polyoxyethylene-polyoxypropylene block copolymers; transesterification products of a polyol with at least one member of the group consisting of glycerides, vegetable oils, hydrogenated vegetable oils, fatty acids and sterols wherein the commonly used oils are castor oil or hydrogenated castor oil, or an edible vegetable oil such as corn oil, olive oil, peanut oil, palm kernel oil, almond oil and the commonly used polyols include glycerol, propylene glycol, ethylene glycol, polyethylene glycol, sorbitol and pentaerythritol; or mixtures thereof.

The solubility enhancing agent that may be employed include, but are not limited to, PEG-20-glyceryl stearate (Capmul® by Abitec), PEG-40 hydrogenated castor oil (Cremophor RH 40® by BASF), PEG-35 castor oil, PEG 6 corn oil (Labrafil® by Gattefosse), lauryl macrogol-32 glyceride (Gelucire 44/14® by Gattefosse), stearoyl macrogol glyceride (Gelucire 50/13® by Gattefosse), polyglyceryl-10 mono dioleate (Caprol PEG 860 by Abitec), propylene glycol oleate (Lutrol OP® by BASF), propylene glycol dioctanoate (Captex® by Abitec), propylene glycol caprylate/caprate (Labrafac® by Gattefosse), glyceryl monooleate (Peceol® by Gattefosse), glycerol monolinoleate (Maisine® by Gattefosse), glycerol monostearate (Capmul® by Abitec), PEG-20 sorbitan monolaurate (Tween 20® by 101), PEG-4 lauryl ether (Brij 30® by ICI), sucrose distearate (Sucroester 7® by Gattefosse), sucrose monopalmitate (Sucroester 15® by Gattefosse), polyoxyethylene-polyoxypropylene block copolymer (Poloxamer or Lutrol® series BASF), polyethylene glycol 660 hydroxystearate, (Solutol® by BASF), sodium lauryl sulphate, sodium dodecyl sulphate, dioctyl sulphosuccinate, L-hydroxypropyl cellulose, hydroxylethylcellulose, hydroxy propylcellulose, propylene glycol alginate, sodium taurocholate, sodium glycocholate, sodium deoxycholate, betains, polyethylene glycol (Carbowax® by DOW), d-α-tocopheryl polyethylene glycol 1000 succinate (Vitamin E TPGS® by Eastman), or mixtures thereof.

The complexing agent that may be employed include, but are not limited to, cyclodextrin class of molecules, such as cyclodextrins containing from six to twelve glucose units, especially, alpha-cyclodextrin, beta-cyclodextrin, gamma-cyclodextrin, or their derivatives, such as hydroxypropyl beta cyclodextrins, or mixtures thereof. The complexing agents may also include cyclic amides, hydroxyl benzoic acid derivatives as well as gentistic acid. In this complexation process, a hydrophilic polymer may be additionally added to further enhance the solubility along with the complexing agent.

In the composition of the present invention, the direct thrombin inhibitor and one or more solubility enhancing agents may be employed in different ratios. The selected ratio depends upon the desired improvement in solubility and the type of solubility enhancing agents employed. It is contemplated within the scope of the invention that the ratio of direct thrombin inhibitor to solubility enhancing agents may range from about 50:1 to about 1:50. In one embodiment, the ratio of direct thrombin inhibitor to solubility enhancing agent is from about 20:1 to about 1:20. In another embodiment, the ratio of direct thrombin inhibitor to solubility enhancing agent is from about 10:1 to about 1:10.

In one embodiment in the composition, the direct thrombin inhibitor may be present in the form of physical blend, solid dispersion, solid solution or complex with the solubility enhancing agent. Different processes may be employed to prepare the composition of the direct thrombin inhibitor with the solubility enhancing agents. It is contemplated within the scope of the invention that the processes for preparing solubilized direct thrombin inhibitor may include, but not limited to, solubilization using melt granulation, solvent treatment, wet granulation, physical mixing or spray drying of the dissolved direct thrombin inhibitor in a solvent with a solubility enhancing agent, melt extrusion, jet milling, shock cooling and the like or combinations thereof. In the case of melt granulation, the solubility enhancing agent is melted. The direct thrombin inhibitor is then added and mixed with the molten mass, and allowed to solidify to form granules which are then separated from each other. In another embodiment the solubility enhancing agents are melted. The direct thrombin inhibitor is then added and mixed with the molten mass. This blend is further mixed with diluents capable of converting this semisolid mass into dry powder. Non limiting examples of such drying agents include celluloses such as microcrystalline cellulose, silicon dioxide, silicates, magnesium aluminium silicate etc. In another illustrative embodiment of this system, the direct thrombin inhibitor is granulated using a molten solubility enhancing agent. In some cases, the direct thrombin inhibitor and the solubility enhancing agent both may be melted together and congealed to room temperature. In using a solvent treatment method, either the solubility enhancing agents or the direct thrombin inhibitor, or both, are dissolved in a solvent which is then evaporated or spray dried. The resultant mass is a blend of direct thrombin inhibitor and solubility enhancing agent, such that the solubility of the direct thrombin inhibitor is increased. The solvent employed in this system may be aqueous or non-aqueous. In the case of physical mixing, the direct thrombin inhibitor and the solubility enhancing agent are preferably intimately dry-mixed using a low shear granulator, a V-blender, or a high shear granulator. In the complexation method, complex of direct thrombin inhibitor can be prepared using different techniques such as ball milling, solvent evaporation method which includes, but is not limited to, spray drying and lyophilization process, slurry method, and paste method. It is contemplated within the scope of the invention that a combination of aforementioned processes can be employed. For example, a combination of hot melt process, physical mixing, and solvent treatment method may be employed. In this case, the direct thrombin inhibitor may be initially granulated with one or more molten solubility enhancing agents, which can be further treated with the same or different solubility enhancing agents in a solvent or with simple physical mixing or vice versa. It is also contemplated within the scope of the invention that any process known in the art suitable for making pharmaceutical compositions in general may be employed for the purpose of this invention.

In one embodiment suitable permeation enhancers that may be employed in the compositions of the present invention include, but are not limited to, surfactants, such as, but not limited to, ionic, non ionic, hydrophilic, amphiphilic, lipophilic surfactants; bile salts; polysaccharides; synthetic polymers; cyclodextrins; chelators and the like or any combinations thereof. Suitable ionic surfactants, include, but are not limited to, cetylpyridinium chloride, alkylammonium salts, sodium lauryl sulfate, sodium laureate, fusidic acid salts, fatty acid derivatives of amino acids, oligopeptides, polypeptides, glyceride derivatives of amino acids, lecithins or hydrogenated lecithins, lysolecithins or hydrogenated lysolecithins, phospholipids or derivatives thereof, lysophospholipids or derivatives thereof, carnitine fatty acid ester salts, salts of alkylsulfates, fatty acid salts, sodium docusate, acyl lactylates, mono- or di-acetylated tartaric acid esters of mono- or di-glycerides, succinylated mono- or di-glycerides, citric acid esters of mono- or di-glycerides, and the like or mixtures thereof. Suitable nonionic surfactants, include, but are not limited to, polyethylene glycol sorbitan fatty acid esters, polyethylene glycol fatty acid monoesters, polyethylene glycol fatty acid diesters, hydrophilic trans-esterification products of alcohols or polyols with at least one member of the group consisting of natural and/or hydrogenated oils such as castor oil or hydrogenated castor oil, or an edible vegetable oil such as corn oil, olive oil, peanut oil, palm kernel oil, almond oil; polysorbate-80, diethylene glycol octadecyl ether, and the like or mixtures thereof. Suitable bile salts include, but are not limited to, bile salts not limited to sodium glycodeoxycholate, sodium glycocholate, sodium taurodeoxycholate, sodium taurocholate and the like or mixtures thereof. Suitable polysaccharides include, but are not limited to, chitosan and the like or mixtures thereof. Suitable synthetic polymers include, but are not limited to carbopol, carbomer; fatty acids not limited to oleic acid, caprylic acid; thiolated polymers of polyacrylates not limited to thiolated sodium carboxy methyl cellulose and the like or mixtures thereof. Suitable chelators, include but are not limited to ethylenediaminetetraacetic acid, sodium citrate and the like or mixtures thereof.

In another embodiment the controlled release of the present invention with improved bioavailability optionally comprise P-glycoprotein inhibitors. The P-glycoprotein inhibitors that may be included in the compositions of the present invention include, but are not limited to, curcumin; phenyl cinnamate; coumarin; beta-amyrin cinnamate; apiole; bergamotin; caffeine; morin; nariturin; piperine; qurcetin; slavironin; silybin; theobromin; vanillin; vanillyl-N-nonlymine; surfactants such as, but not limited to, tocopherol polyethylene glycol succinic acid esters (TPGS) not limited to those that are commercially under the trade name Vitamin E TPGS; reaction products of a natural or hydrogenated castor oil and ethylene oxide not limited to those that are available commercially under the trade name Cremophor® EL, Cremophor® RH40; polyoxyethylene-sorbitan-fatty acid esters not limited to those available commercially under the trade name Tween®; polyoxyethylene-polyoxypropylene co-polymers and block co-polymers or, poloxamers not limited to those available commercially under the trade name Pluronic®; transesterified, polyoxyethylated caprylic-capric acid glycerides not limited to those available commercially under the trade name Labrasol®, and the like or combinations thereof.

In a further embodiment the controlled release of the present invention with improved bioavailability comprise swelling enhancers. Swelling enhancers help the swelling agents to swell rapidly to a large extent resulting in a dramatic increase in the size of the tablet. At lower concentrations, these excipients are used as superdisintegrants; however at concentration above 5% w/w these agents function as swelling enhancers and help increase the size of the dosage form. According to the present invention, swelling enhancers that may be incorporated include, but are not limited to, low-substituted hydroxypropyl cellulose, microcrystalline cellulose, cross-linked sodium or calcium carboxymethyl cellulose, cellulose fiber, cross-linked polyvinyl pyrrolidone, cross-linked polyacrylic acid, cross-linked amberlite resin, alginates, colloidal magnesium-aluminum silicate, corn starch granules, rice starch granules, potato starch granules, pregelatinised starch, sodium starch glycolate and sodium carboxymethyl starch. In one embodiment matrix osmogents, such as but not limited to, dextrose, mannitol, sodium chloride and the like or combinations thereof are employed as swelling enhancers.

The amount of swelling enhancers used in the dosage forms of the present invention is about 5 to about 90 weight percent. In one embodiment, the amount of the swelling enhancer is about 10 to about 70 weight percent. In another embodiment, the amount of the swelling enhancer is about 15 to about 50 weight percent. In one embodiment, the dosage forms according to the present invention include at least one swelling agent and a swelling enhancer. When a combination of swelling agent and swelling enhancer is employed for gastric-retention, it allows a rapid and dramatic increase in the size of the tablets. Such a combination may be employed which allows rapid swelling and maintenance of integrity by polymeric network formed upon swelling of the polymer(s).

Gas generating agents aid in the formation of highly porous, preferably honeycombed structure and enhance the buoyancy of the formulation. The gas generating agent employed in the present invention is selected from, but not limited to, alkali and alkaline-earth metal carbonates and bicarbonates such as sodium bicarbonate, sodium glycine carbonate, potassium bicarbonate, ammonium bicarbonate, sodium bisulfite, sodium metabisulfite, sodium carbonate, potassium carbonate and the like. In one embodiment, the gas generating, agent is sodium bicarbonate. The pharmaceutical composition can further optionally comprise an acid source. The acid source may be, but is not limited to, citric acid, maleic acid, oxalic acid, malonic acid, succinic acid, glutaric acid, adipic acid, fumaric acid, phthalic acid, aspartic acid, glutamic acid, malic acid or tartaric acid. In a dry granulation process, the gas generating agent may be incorporated into the dosage form by blending it into the expanding composition before or after first compaction. In a wet granulation process, it may be provided as an extragranular constituent after wet granulation.

Examples of suitable binders include, but are not limited to, starch, pregelatinized starch, polyvinyl prrolidone, copovidone, cellulose derivatives, such as hydroxypropylmethyl cellulose (HPMC), hydroxypropyl cellulose (HPC) and carboxymethyl cellulose and their salts. Examples of suitable diluents include, but are not limited to, starch, dicalcium phosphate, microcrystalline cellulose, lactose monohydrate, dextrate hydrated and the like. Examples of suitable lubricants include, but are not limited to, magnesium stearate, calcium stearate, stearic acid, talc, and sodium stearyl fumarate. Compositions of the present invention may optionally also include a glidant such as, but not limited to, colloidal silica, silica gel, precipitated silica, or combinations thereof. Suitable disintegrants may optionally be employed in the compositions of the present invention include croscarmellose sodium, crospovidone, sodium starch glycolate, starch or combinations thereof. In one embodiment, suitable pH modifiers may optionally be incorporated in the compositions of the present invention including, but are not limited to tartaric acid, malic acid, fumaric acid, maleic acid, aspartic acid or citric acid.

In a further embodiment the controlled release gastroretentive dosage forms of the present invention may be in the form of a monolithic system, an expanding bilayered or multilayered or in-lay system for oral administration which is adapted to deliver the drug at a predetermined rate. In one embodiment, the direct thrombin inhibitor is incorporated in monolithic matrix type in the controlled release gastroretentive formulation. In another embodiment, the direct thrombin inhibitor is incorporated in the form of a bilayered gastroretentive dosage form that consists of a drug layer and a gastroretentive expanding layer wherein the drug is released at a predetermined rate from the drug layer.

In a further embodiment pharmaceutical controlled release gastroretentive composition in the form of an expanding bilayered system for oral administration is provided to deliver direct thrombin inhibitor from a first layer immediately upon reaching the gastrointestinal tract, and to deliver same or different active, from a second layer, in a sustained manner over a specific time period. The second layer is also adapted to provide expanding nature for the dosage system, thereby making the dosage system have greater retention in the stomach. In yet another embodiment, the controlled release gastroretentive dosage form is in the form of a trilayered system consisting of a drug layer compressed between a first gastroretentive layer and a second gastroretentive layer wherein direct thrombin inhibitor is released at a predetermined rate from the drug layer. In a further embodiment the controlled release gastroretentive dosage form of the present invention comprises direct thrombin inhibitor treated with a release controlling agent. In a further embodiment the controlled release gastroretentive dosage form of the present invention comprises solubilized direct thrombin inhibitor treated with a release controlling agent. The dosage forms of the present invention ensure desired gastroretention and controlled or sustained release of direct thrombin inhibitor thereby improving the oral bioavailability.

In yet another embodiment, the gastroretentive dosage form is in the form of a trilayered system consisting of a drug layer compressed between a gastroretentive layer and a barrier layer wherein direct thrombin inhibitor is released at a predetermined time from the drug layer. The barrier layer acts as a barrier modulating the release and is partially impermeable, for a predeterminable time, to the active ingredient contained in the adjacent drug layer. In one embodiment the excipients employed for the preparation of said barrier layer include but are not limited to, glyceryl monostearate and derivative thereof, semisynthetic glycerides, hydrogenated castor oil, glyceryl palmitostearate, glyceryl behenate, cetyl alcohol, glycerin, cellulose derivatives, ethylcellulose, methylcellulose, sodium carboxymethylcellulose, polymethacrylates, polyvinylpyrrolidone, stearic acid, talc, sodium benzoate, boric acid, polyoxyethylene glycols, colloidal silica and the like. Further for the preparation of barrier layer, plasticizers may be employed such as but not limited to hydrogenated vegetable oils, fatty alcohols, fatty acids, glycerides and triglycerides and their substituted forms, polyoxyethylene glycols and derivatives thereof and the like. In one embodiment the barrier layer may also be characterized in that it can act as a barrier modulating the release and can rapidly swell, i.e. can rapidly increase in volume, and have bioadhesive properties allowing dosage form retention and adhesion to gastrointestinal mucosa.

In a further embodiment controlled release gastroretentive dosage form of the present invention is in the form of an in-lay system comprising a drug containing tablet which is placed in another tablet comprising a blend of excipients that ensure gastric retention. In this system the drug containing tablet is small and is covered from all sides except at least one side with a blend of excipient that ensure the gastric retention.

In yet another illustrative embodiment according to the invention, the controlled release formulation with improved bioavailability may be optionally coated. Surface coatings may be employed for aesthetic purposes or for dimensionally stabilizing the dosage form. The coating may be carried out using any conventional technique employing conventional ingredient. A surface coating can, for example, be obtained using a quick-dissolving film using conventional polymers such as, but not limited to, hydroxypropyl methyl cellulose, hydroxypropyl cellulose, carboxymethyl cellulose, ethyl cellulose, polyvinyl alcohol, poly methacrylates or the like or combinations thereof. Tablets of the present invention may vary in shape including, but not limited to, oval, triangle, almond, peanut, parallelogram, pentagonal. It is contemplated within the scope of the invention that the dosage form can be encapsulated. Tablets in accordance with the present invention may be manufactured using conventional techniques of common tableting methods known in the art such as direct compression, dry granulation, wet granulation and extrusion/melt granulation.

Further, in one embodiment, the present invention provides a process of preparing a controlled release formulation comprising: preparing solubilized direct thrombin inhibitor by treatment with solubility enhancing agent; blending said solubilized direct thrombin inhibitor with at least one release controlling agent, and at least one pharmaceutically acceptable excipient; lubricating the blend to form a lubricated blend; compressing the blend to form a monolithic tablet. In another embodiment, the present invention provides a process of preparing a controlled release gastroretentive formulation comprising: preparing solubilized direct thrombin inhibitor by treatment with solubility enhancing agent; blending said solubilized direct thrombin inhibitor with at least one release controlling agent, at least one swelling agent and at least one pharmaceutically acceptable excipient; lubricating the blend to form a lubricated blend; compressing the blend to form a monolithic tablet. Furthermore, the present invention also provides a process of preparing a controlled release gastroretentive dosage form of direct thrombin inhibitor comprising: preparing solubilized direct thrombin inhibitor by treatment with solubility enhancing agent; blending said solubilized direct thrombin inhibitor with at least one release controlling agent and at least one pharmaceutically acceptable excipient, lubricating the blend to form drug layer blend; blending at least one swelling agent, at least one pharmaceutically acceptable excipient, lubricating the blend to form a gastroretentive layer blend; and compressing the drug layer and the gastroretentive layer to form a bilayer tablet.

The controlled release gastroretentive dosage form of the present invention that may be coated/uncoated, single layer or multilayered composition, gradually swells upon contact with the gastric fluid. The time taken for swelling may vary from about 15 minutes to about 4 hours. In one embodiment, the time taken for swelling is within about 15 minutes to about 3 hours. In another embodiment, the time taken for swelling is within about 15 minutes to about 2 hours.

Two dimensions of the dosage form namely length and width expand to more than about 8 mm after swelling within 2 hours in media simulating typical gastric environment (0.1N hydrochloric acid). In one embodiment, the length and width of the dosage form expand to more than about 10 mm after swelling within 2 hours in media simulating typical gastric environment (0.1N hydrochloric acid). In another embodiment, the length and width of the dosage form expand to more than about 12 mm after swelling within 2 hours in media simulating typical gastric environment (0.1N hydrochloric acid). The present invention provides controlled release formulations of direct thrombin inhibitor that are more than about 1 to about 4 times more bioavailable than the conventional immediate release dosage forms. The controlled release formulations according to the present invention allow for controlled release of direct thrombin inhibitor. In one embodiment the direct thrombin inhibitor is released over a period of more than about 4 hours. In a further embodiment the direct thrombin inhibitor is released over a period of about 6 hours. In one embodiment the direct thrombin inhibitor is released over a period of about 8 hours. In another embodiment the direct thrombin inhibitor released over a period of about 12 hours. In another embodiment the direct thrombin inhibitor released over a period of about 24 hours.

Further, within the purview of the present invention, are included formulations that comprise a combination of direct thrombin inhibitor with other drugs or active agents which may be delivered in an immediate release or modified release manner, including not limited to, atorvastatin, dipyridamole, mopidamole and the like or combinations thereof.

In a further embodiment is provided the use of pharmaceutical composition of direct thrombin inhibitor for the manufacture of a medicament for reducing the risk of stroke and systemic embolism in patients with non-valvular atrial fibrillation and/or preventing venous thromboembolic events in adult patients who have undergone elective total hip replacement surgery or total knee replacement surgery. Further, the present invention provides a method for reducing the risk of stroke and systemic embolism in patients with non-valvular atrial fibrillation and/or preventing venous thromboembolic events in adult patients who have undergone elective total hip replacement surgery or total knee replacement surgery, comprising administering to the subject in need thereof pharmaceutical compositions of direct thrombin inhibitors of the present invention.

While the present invention has been described in terms of its specific embodiments, certain modifications and equivalents will be apparent to those skilled in the art and are intended to be included within the scope of the present invention. The invention is further illustrated by the following examples, which are for illustrative purposes and should not be construed as limiting the scope of the invention in any way.

### EXAMPLES

### Example 1

### Controlled Release Gastroretentive Tablet of Dabigatran Etexilate Mesylate

Preparation of Gastroretentive Tablet of Dabigatran Etexilate Mesylate

TABLE 1
Composition of gastroretentive tablet of dabigatran etexilate mesylate

| Ingredients | mg/tablet |
|---|---|
| Dabigatran etexilate mesylate equivalent to 75 mg of | 86.48 |
| dabigatran etexilate | |
| Stearoyl macrogol glyceride, USP/NF (Gelucire 50/13 ®) | 80 |
| Vitamin E TPGS, USP/NF (d-α-tocopheryl polyethylene | 40 |
| glycol 1000 succinate) | |
| Polyethylene oxide, USP/NF | 180 |
| Hydroxy propyl methyl cellulose, USP/NF (Methocel K100M) | 90 |
| Microcrystalline cellulose, USP/NF | 120.52 |
| Povidone, USP/NF | 32 |
| Crospovidone, USP/NF | 200 |
| Sodium bicarbonate, USP/NF | 40 |
| Citric acid, USP/NF | 16 |
| Magnesium stearate, USP/NF | 10 |
| Total | 895 |

Procedure:
Stearoyl macrogol glyceride and Vitamin E TPGS was melted and dabigatran etexilate mesylate, part of microcrystalline cellulose was added to the same. The mass was then sized and screened to obtain granules of dabigatran etexilate mesylate. These granules were then blended with other excipients except lubricant, then lubricated and compressed to form gastroretentive tablet.

### Example 2

### Controlled Release Gastroretentive Tablet of Dabigatran Etexilate Mesylate

A. Preparation of Controlled Release Drug Layer

TABLE 2
Composition of controlled release drug layer

| Ingredients | mg/tablet |
|---|---|
| Dabigatran etexilate mesylate equivalent to 110 mg of | 126.83 |
| dabigatran etexilate | |
| Poloxamer 407, USP/NF | 120 |
| Hydroxy propyl methyl cellulose, USP/NF (Methocel K4M) | 40 |
| Microcrystalline cellulose, USP/NF | 110.17 |
| Povidone, USP/NF | 20 |
| Magnesium stearate, USP/NF | 8 |
| Weight of drug layer | 425 |

Procedure:
All the excipients except the lubricant were blended. This blend was then lubricated to form lubricated drug layer blend.

B. Preparation of Gastroretentive Layer

TABLE 3
Composition of gastroretentive layer

| Ingredients | mg/tablet |
|---|---|
| Hydroxy propyl methyl cellulose, USP/NF (Methocel K100M) | 175 |
| Polyethylene oxide, USP/NF | 175 |
| Crospovidone, USP/NF | 175 |
| Microcrystalline cellulose, USP/NF | 60 |
| Lactose, USP/NF | 20 |
| Povidone, USP/NF | 30 |
| Sodium bicarbonate, USP/NF | 40 |
| Citric acid, USP/NF | 15 |
| Magnesium stearate, USP/NF | 10 |
| Weight of gastroretentive layer | 700 |

Procedure:
All ingredients except lubricant were dry mixed. The blend was then lubricated using magnesium stearate to form the gastroretentive layer blend.

A bilayer gastroretentive tablet of dabigatran etexilate mesylate was prepared by compressing the drug layer blend and the gastroretentive layer blend.

We claim:

1. A controlled release formulation comprising at least one direct thrombin inhibitor, at least one release controlling agent and at least one pharmaceutically acceptable excipient.

2. The controlled release formulation of claim 1, wherein the direct thrombin inhibitor is dabigatran, argatroban, inogatran, melagatran, ximelagatran, hirudin, bivalirudin, lepirudin, or desirudin.

3. The controlled release formulation of claim 2, wherein the direct thrombin inhibitor is in the form of a free acid, a free base, a pharmaceutically acceptable prodrug, a pharmaceutically acceptable salt, a pharmaceutically acceptable salt of prodrug, an active metabolite, a polymorph, a precursor, a derivative, an analog, an amorphous form, a diastereomer, a diastereomeric mixtures, a solvate, a hydrate, an enantiomer, an optical isomer, a tautomer, a racemic mixture or any combination thereof.

4. The controlled release formulation of claim 2, wherein the direct thrombin inhibitor is dabigatran etexilate mesylate.

5. The controlled release formulation of claim 1, wherein the release controlling agent is polymeric release controlling agent, non-polymeric release controlling agent or any combination thereof.

6. The controlled release formulation of claim 5, wherein the polymeric release controlling agent is cellulose derivative, saccharide or polysaccharide, poly(oxyethylene)-poly(oxypropylene) block copolymer, vinyl derivative or polymer or copolymer thereof, polyalkylene oxide and derivative thereof, maleic copolymer, acrylic acid derivative, or any combination thereof.

7. The controlled release formulation of claim 5, wherein the non-polymeric release modifier is a fatty acid, long chain alcohol, fat, oil, wax, phospholipid, eicosonoid, terpene, steroid, resin or any combination thereof.

8. The controlled release formulation of claim 1, wherein the formulation further comprises at least one swelling agent.

9. The controlled release formulation of claim 8, wherein the swelling agent is at least one hydrophilic polymer.

10. The controlled release dosage from of claim 9, wherein the hydrophilic polymer is polyalkylene oxide, cellulosic polymer, acrylic acid and methacrylic acid polymer or ester thereof, polyethylene oxide, maleic anhydride polymer; polymaleic acid, poly(acrylamide); poly(olefinic alcohol), poly(N-vinyl lactam), polyol, polyoxyethylated saccharide, polyoxazoline, polyvinylamine, polyvinylacetate, polyimine, starch and starch-based polymer, polyurethane hydrogel, chitosan, polysaccharide gum, alginate, zein, shellac-based polymer, polyacrylic acid, maltodextrin, pre-gelatinized starch, polyvinyl alcohol, or any combination thereof.

11. The controlled release formulation of claim 1, wherein the pharmaceutically acceptable excipient is a solubility enhancing agent, p-glycoprotein inhibitor, swelling enhancer, permeation enhancer, binder, lubricant, diluent, disintegrant, glidant, stabilizer, pH modifier, preservative, colorant or any combination thereof.

12. The controlled release formulation of claim 1, wherein the formulation is in the form of a matrix controlled-release system, coated controlled release system, coated-matrix controlled release system, osmotic controlled-release system, multiparticulate controlled-release system, non-gastroretentive controlled release system.

13. The controlled release formulation of claim 1, wherein the formulation is in the form of gastroretentive dosage form.

14. The controlled release formulation of claim 13, wherein the gastroretentive dosage form is in the form of a monolithic system, an expanding bilayered or multilayered or in-lay system.

15. The controlled release formulation of claim 14, wherein the bilayered gastroretentive dosage form comprises (a) a direct thrombin inhibitor layer and (b) a gastroretentive layer.

16. The controlled release formulation of claim 15, wherein the direct thrombin inhibitor layer comprises at least one direct thrombin inhibitor, at least one release controlling agent, at least one pharmaceutically acceptable excipient, and optionally at least one swelling agent; and the gastroretentive layer comprises at least one swelling agent and at least one pharmaceutically acceptable excipient.

17. The controlled release formulation of claim 13, wherein the dosage form is retained in the upper gastrointestinal tract for a time period of about 30 min to about 12 hours.

18. The controlled release formulation of claim 1, wherein the dosage form releases at least one direct thrombin inhibitor over a period of up to about 24 hours.

19. The controlled release formulation of claim 1, wherein the formulation further comprises one or more active agents.
